(19) United States
(12) Patent Application Publication
(10) Pub. No.: US 2017/0216170 A1
(43) Pub. Date: Aug. 3, 2017

(54) ANTIPERSPIRANTS COMPRISING ALUMINUM SESQUICHLOROHYDRATE AND TRIETHYL CITRATE

(71) Applicant: Henkel AG & Co. KGaA, Duesseldorf (DE)

(72) Inventor: Thomas Doering, Dormagen (DE)

(73) Assignee: Henkel AG & Co. KGaA, Duesseldorf (DE)

(21) Appl. No.: 15/486,730

(22) Filed: Apr. 13, 2017

Related U.S. Application Data
(63) Continuation of application No. PCT/EP2015/070436, filed on Sep. 8, 2015.

(30) Foreign Application Priority Data
Oct. 15, 2014 (DE) 102014220918.8

(51) Int. Cl. A61K 8/26; A61Q 15/00; A61K 8/34; A61K 8/06; A61K 8/37

(52) U.S. Cl. CPC A61K 8/26; A61K 8/062; A61K 8/37; A61K 8/34; A61Q 15/00; A61K 2800/30

(57) ABSTRACT

The present invention relates to a highly effective antiperspirant cosmetic agent with low pH in the form of an aqueous emulsion which includes aluminum sesquichlorohydrate and triethyl citrate.

### FIELD OF THE INVENTION

The present invention generally relates to an antiperspirant cosmetic agent with low pH in the form of an aqueous emulsion which includes aluminum sesquichlorohydrate and triethyl citrate.

The present invention also relates to a non-therapeutic cosmetic method for treating the human body, in particular for preventing and/or reducing the perspiration of the body, in which the antiperspirant agent according to the invention is applied to the skin, in particular to the skin of the underarms.

The present invention also relates to the use of a combination of aluminum sesquichlorohydrate and ethyl citrate, these being present in an aqueous emulsion having a pH of from 3.5 to 4.0, for reducing and/or preventing perspiration, in particular underarm perspiration.

### BACKGROUND OF THE INVENTION

The washing, cleaning and care of one's body is a basic human need, and modern industry is constantly attempting to meet these human needs in many ways. The lasting elimination or at least reduction in body odor is particularly important for daily hygiene. In the prior art, numerous specific deodorizing or antiperspirant body care agents are known which have been developed for use in body regions having a high density of sweat glands, in particular in the underarm area. These are prepared in various administration forms, for example as a powder, in stick form, as an aerosol spray, pump spray, liquid and gel-like roll-on application, cream, gel, and as an impregnated flexible substrate (deodorant wipes).

Cosmetic antiperspirants usually include at least one oil or one fatty substance, a fragrance component or a perfume, and at least one antiperspirant salt.

On the one hand, the antiperspirant salts used in antiperspirants reduce the body's sweat secretion by temporarily narrowing or blocking the efferent ducts of the sweat glands, so that the amount of sweat can be reduced by around 20 to 60 percent. On the other hand, due to their antimicrobial action, they have an additional deodorizing effect.

Activated basic aluminum and aluminum-zirconium halides, as described in the documents EP 0308937, EP 0183171, U.S. Pat. No. 4,359,456 and EP 0191628, are usually used as the antiperspirant salt. In addition, use can also be made of aluminum and aluminum-zirconium halides which are stabilized with organic acids as complex ligands, as disclosed for example in the documents U.S. Pat. No. 3,542,919, U.S. Pat. No. 3,553,316, U.S. Pat. No. 3,991,176 and WO 2005/092795.

WO 2014/083175 and US 2005/0163737 disclose, besides other aluminum salts, the use of various aluminum chlorohydrates and derivatives thereof as antiperspirant constituents of antiperspirants, including, among others, also aluminum sesquichlorohydrate. Regular aluminum chlorohydrate usually has a molar Al/Cl ratio of around 1.9:1 to 2.1:1. Aluminum sesquichlorohydrate has a lower molar Al/Cl ratio, often of around 1.5:1 to 1.8:1. In aqueous solution, aluminum sesquichlorohydrate reacts in a more acidic manner than aluminum chlorohydrate.

Aluminum chlorohydrate and other aluminum salts which are used as components having an antiperspirant effect usually require high dosages of around 20% by weight of the antiperspirant agent. The consequences are undesirable residues on the skin and stains on fabrics, as well as a sticky feel and skin intolerances.

The pH of the skin surface is typically around pH 5.5. So as not to further increase the irritant effect of aluminum salts such as aluminum chlorohydrate on the skin surface, the pH of commercially available antiperspirant compositions is usually adjusted to a range greater than pH 4.0 to around 4.5, that is to say a pH which is not too different from the pH of the skin surface.

### BRIEF SUMMARY OF THE INVENTION

An antiperspirant cosmetic agent in the form of an aqueous emulsion which includes:
aluminum sesquichlorohydrate and
triethyl citrate,
   wherein the cosmetic agent has a pH of from 3.5 to 4.0.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The problem addressed by the present invention was that of providing an antiperspirant agent which enables a low dose of an aluminum salt as an antiperspirant active ingredient, wherein a very good antiperspirant effect is achieved and skin intolerances and residues on skin and stains on fabrics can be avoided as far as possible.

It has now surprisingly been found according to this invention that this can be achieved by an antiperspirant cosmetic agent which includes a combination of aluminum sesquichlorohydrate and triethyl citrate, wherein the agent is formulated as an aqueous emulsion and has a pH of from 3.5 to 4.0.

With regard to preferred embodiments of the method according to the invention and of the use according to the invention, what is stated in relation to the agents according to the invention applies mutatis mutandis.

It has surprisingly been found that a particularly good antiperspirant effect can be achieved by combining the specific antiperspirant active ingredient aluminum sesquichlorohydrate with triethyl citrate (INCI: Triethyl Citrate). This enables aluminum sesquichlorohydrate to be incorporated in a relatively small amount of for example 8 to 12% by weight, based on the total weight of the antiperspirant agent, without taking into account any propellant that may be present. Said two components evidently interact in a synergistic manner. Without wishing to be bound to one theory, it is suspected that particularly effective complexes of aluminum are formed with this combination.

When the antiperspirant agent according to the invention was applied, very good skin compatibility, few residues and stains on the skin and fabrics and an improved, less sticky feel on the skin were observed.

Furthermore, when the cosmetic agent according to the invention was applied to the skin surface, a lowering of the pH of the skin was observed. In general, when the cosmetic agent according to the invention is applied to the skin, a reduction in the pH of the skin by around 0.2 to 0.7 is observed within 60 minutes.

The components aluminum sesquichlorohydrate and triethyl citrate have such a good synergistic effect that, according to embodiments of the invention, no further components having an antiperspirant effect need be included. Such compositions which include no other components having an antiperspirant effect are expressly encompassed according to the invention.

In embodiments of the invention, the antiperspirant cosmetic agent includes no ethylhexylglycerin. In further embodiments of the invention, the antiperspirant cosmetic agent includes no dipropylene glycol. Dipropylene glycol can impair the antiperspirant effect of the cosmetic composition of the invention and is therefore not included in a preferred embodiment. In further embodiments of the invention, no dipropylene glycol and no propylene glycol are included in the cosmetic agent.

Of course, however, the invention also encompasses embodiments in which further components having an antiperspirant effect are included in order where necessary to further enhance the antiperspirant effect of the cosmetic agent according to the invention. Other possible constituents of the cosmetic agent according to the invention will be explained below.

One necessary constituent of the antiperspirant agent according to the invention is aluminum sesquichlorohydrate. Aluminum sesquichlorohydrate can be described in general by the formula Al₂Clₐ(OH)₆₋ₐ, for example Al₂(OH)₄Cl_{1.5}.xH₂O, these formulae indicating the approximate composition. According to the invention, a is preferably in the range from 1.33 to around 1.1, so that the aluminum sesquichlorohydrate then has a molar Al/Cl ratio of from around 1.5:1 to around 1.8:1.

The aluminum sesquichlorohydrate is preferably included in the antiperspirant agent according to the invention in a proportion of from 5.0 to 15.0% by weight, more preferably from 6.0 to 14.0% by weight, even more preferably from 7.0 to 13.0% by weight and yet more preferably from 8.0 to 12.0% by weight, wherein the figures specified in % by weight relate in each case to the total weight of the agent according to the invention, without taking into account any propellant that may be present.

Another necessary constituent of the antiperspirant agent according to the invention is triethyl citrate. Triethyl citrate is a known deodorant active ingredient. Preferred agents according to the invention include from 0.1 to 7.0% by weight triethyl citrate, more preferably from 0.5 to 5.0% by weight, even more preferably from 0.5 to 3.0% by weight, most preferably from 1.0 to 2.0% by weight, in each case based on the weight of the composition, without taking into account any propellant that may be present.

The cosmetic agent according to the invention includes the components aluminum sesquichlorohydrate and triethyl citrate in combination preferably in the following proportions:

aluminum sesquichlorohydrate in an amount of from 5.0 to 15.0% by weight, more preferably from 8.0 to 12.0% by weight, and triethyl citrate in an amount of from 0.1 to 7.0% by weight, more preferably from 0.5 to 2.0% by weight, wherein the figures specified in % by weight relate in each case to the total weight of the agent according to the invention, without taking into account any propellant that may be present.

Commercially available antiperspirant agents which include aluminum salts as active components have, as mentioned, a pH greater than 4.0, wherein usually a higher pH is desired in order to come closer to the pH of the skin surface and thus to minimize skin irritation.

It was therefore surprising that, by combining the components triethyl citrate and aluminum sesquichlorohydrate in an aqueous emulsion having a pH of from 3.5 to 4.0, it was possible on the one hand to achieve an excellent antiperspirant effect and at the same time to minimize skin intolerances.

The pH of the aqueous emulsion or of the antiperspirant agent in the present invention is preferably from 3.5 to 3.95, more preferably from 3.6 to 3.9, even more preferably from 3.7 to 3.8. According to the invention, the pH is measured using the pH meter pH730 from inoLab under normal conditions.

The cosmetic agent according to the invention exists as an aqueous emulsion, preferably as an oil-in-water emulsion. In this case, the agent according to the invention is preferably sprayed as a propellant-free pump spray or squeeze spray or applied as a roll-on.

In another preferred administration form, the antiperspirant cosmetic agent exists as a water-in-oil emulsion. This may be in particular a sprayable water-in-oil emulsion which can be sprayed by means of a propellant.

The oil component is a cosmetic oil. Where a cosmetic oil is mentioned in this invention, this always means a cosmetic oil which is not a fragrance and is not an essential oil, is liquid under normal conditions and is immiscible with water. Suitable cosmetic oils are volatile cosmetic oils and non-volatile cosmetic oils.

According to the invention, the term “volatile cosmetic oil” refers to cosmetic oils which at 20° C. and an ambient pressure of 1013 hPa have a vapor pressure of from 2.66 Pa to 40,000 Pa (0.02 to 300 mmHg), preferably from 10 to 12,000 Pa (0.1 to 90 mmHg), more preferably from 13 to 3000 Pa (0.1 to 23 mmHg), in particular from 15 to 500 Pa (0.1 to 4 mmHg).

In addition, in the context of the present invention, the term “non-volatile cosmetic oils” will be understood to mean cosmetic oils which at 20° C. and an ambient of 1013 hPa have a vapor pressure of less than 2.66 Pa (0.02 mmHg).

According to one preferred embodiment of the present invention, the cosmetic oil which is liquid at 20° C. and 1013 hPa is selected from the group consisting of

(i) volatile cyclic silicone oils, in particular cyclotrisiloxane, cyclotetrasiloxane, cyclopentasiloxane and cyclohexasiloxane, and linear silicone oils having 2 to 10 siloxane units, in particular hexamethyldisiloxane, octamethyltrisiloxane, decamethyltetrasiloxane;
(ii) volatile non-silicone oils, in particular liquid paraffin oils and isoparaffin oils, such as isodecane, isoundecane, isododecane, isotridecane, isotetradecane, isopentadecane, isohexadecane and isoeicosane;
(iii) non-volatile silicone oils, in particular higher-molecular-weight linear polyalkylsiloxanes;
(iv) non-volatile non-silicone oils, in particular the esters of linear or branched saturated or unsaturated C₂-C₃₀ fatty alcohols with linear or branched saturated or unsaturated C₂-C₃₀ fatty acids, which may be hydroxylated, the C₈-C₂₄ fatty alcohol esters of monovalent or polyvalent C₂-C₇ hydroxycarboxylic acids, the branched saturated or unsaturated C₆-C₃₀ fatty alcohols, the mono-, di- and triglycerides of linear or branched, saturated or unsaturated, optionally hydroxylated C₆-C₃₀ fatty acids, the dicarboxylic acid esters of linear or branched C₂-C₁₀ alkanols, the addition products of ethylene oxide and/or propylene oxide with monovalent or polyvalent C₃-C₂₂ alkanols, which may optionally be esterified, the symmetrical, asymmetrical or cyclic esters of carbonic acid with fatty alcohols, the esters of dimers of unsaturated C₁₂-C₂₂ fatty acids with monovalent, linear, branched and cyclic C₂-C₁₈ alkanols or C₂-C₆ alkanols, the benzoic acid esters of linear or branched C₈-C₂₂ alkanols, such as benzoic acid C₁₂-C₁₅ alkyl ester and isostearyl benzoate and octyldodecyl benzoate, the synthetic hydrocarbons, such as polyisobutene and polydecene, the alicyclic hydrocarbons; and
(v) mixtures thereof.

The use of volatile silicone oils and volatile non-silicone oils in the antiperspirant cosmetic agents according to the invention results in a drier skin feel and in a faster release of the antiperspirant aluminum salt.

The cyclic volatile silicone oils which can be used in the context of the invention have at 20° C. and an ambient pressure of 1013 hPa a vapor pressure of from 13 to 15 Pa (0.1 mmHg). According to the invention, the linear volatile silicone oil used can also be a low-molecular-weight phenyl trimethicone having a vapor pressure of around 2000 Pa (15 mmHg) at 20° C. and an ambient pressure of 1013 hPa. However, due to the high persistence of cyclodimethicones in the environment, it may be preferred according to the invention if from 0 to less than 1% by weight, preferably from 0 to less than 0.1% by weight, cyclic volatile silicone oils are used in the antiperspirant cosmetic agents according to the invention.

According to the invention, use will preferably be made of volatile non-silicone oils in the form of C₁₀₋₁₃ isoparaffin mixtures having a vapor pressure of from 10 to 400 Pa (0.08 to 3 mmHg), preferably from 13 to 100 Pa (0.1 to 0.8 mmHg), at 20° C. and an ambient pressure of 1013 hPa. It is preferred in the context of the present invention if the volatile C₈-C₁₆ isoparaffin is included in a total amount of from 1 to 60% by weight, preferably from 3 to 45% by weight, more preferably from 5 to 40% by weight, in particular from 8 to 20% by weight, based on the total weight of the antiperspirant cosmetic agent. Of course, it is also possible to formulate antiperspirant cosmetic agents according to the invention with a low proportion of volatile oils, that is to say with from 0.5 to 15% by weight of volatile oils, based on the total weight of the antiperspirant cosmetic agent, or even without volatile oils.

In order to mask insoluble constituents, such as talc or antiperspirant aluminum salts that have dried onto the skin, it may be preferred according to the invention if the antiperspirant cosmetic agents include a non-volatile silicone oil and/or a non-volatile non-silicone oil.

In this connection, antiperspirant cosmetic agents which are preferred according to the invention include at least one ester of the linear or branched saturated or unsaturated fatty alcohols having 2 to 30 carbon atoms with linear or branched saturated or unsaturated fatty acids having 2 to 30 carbon atoms, which may be hydroxylated, in a total amount of from 1 to 30% by weight, preferably from 5 to 26% by weight, more preferably from 9 to 24% by weight, based on the total weight of the antiperspirant cosmetic agent.

In the context of the present invention, the non-volatile silicone oils used may be linear polyalkylsiloxanes having a kinematic viscosity at 25° C. of from 5 to 2000 cSt, in particular linear polydimethylsiloxanes having a kinematic viscosity at 25° C. of from 5 to 2000 cSt, preferably from 10 to 350 cSt, in particular from 50 to 100 cSt. The abovementioned non-volatile silicone oils are obtainable under the trade names Dow Corning© 200 and Xiameter PMX from Dow Corning and Xiameter respectively. Other preferred non-volatile silicone oils are phenyl trimethicones having a kinematic viscosity at 25° C. of from 10 to 100 cSt, preferably from 15 to 30 cSt, as well as cetyl dimethicones.

According to the invention, preference is also given to the use of mixtures of the abovementioned cosmetic oils, in particular of non-volatile and volatile cosmetic oils, since in this way parameters such as skin feel, visibility of the residue and stability of the antiperspirant cosmetic agent according to the invention can be adjusted and the agent can thus be better adapted to consumer requirements.

In the context of the present invention, it is preferred if the cosmetic oil which is liquid at 20° C. and 1013 hPa is included in a total amount of from 1 to 95% by weight, preferably from 10 to 85% by weight, more preferably from 20 to 75% by weight, even more preferably from 35 to 70% by weight, based on the total weight of the antiperspirant cosmetic agent.

However, it may also be preferred if the cosmetic oil which is liquid at 20° C. and 1013 hPa is included in a total amount of from 0.2 to 70% by weight, preferably from 2 to 60% by weight, more preferably from 3 to 50% by weight, even more preferably from 5 to 35% by weight, in particular from 8 to 20% by weight, based on the total weight of the antiperspirant cosmetic agent.

It should be noted at this point, unless indicated otherwise, that the figures specified in % by weight relate in each case to the total weight of the agent according to the invention, without taking into account any propellant that may be present.

The terms “(antiperspirant or cosmetic) agent according to the invention” and “composition according to the invention” are used synonymously in the present invention.

In the context of the present invention, “normal conditions” are a temperature of 20° C. and a pressure of 1013.25 mbar. Specified melting points also relate to a pressure of 1013.25 mbar.

The antiperspirant cosmetic agents according to the invention may additionally include further auxiliaries. Preferably, the antiperspirant cosmetic agents according to the invention include at least one further auxiliary selected from the group consisting of (i) emulsifiers and/or surfactants; (ii) hydrogel formers; (iii) chelating agents; (iv) deodorant active ingredients; (v) monovalent and/or polyvalent alcohols and/or polyethylene glycols; (vi) skin-cooling active ingredients; (vii) propellants; (viii) thickeners, and (ix) mixtures thereof

According to the invention, suitable emulsifiers and surfactants are preferably selected from anionic, cationic, nonionic, and amphoteric, in particular ampholytic and zwitterionic emulsifiers and surfactants. Surfactants are amphiphilic (bifunctional) compounds which consist of at least one hydrophobic moiety and at least one hydrophilic moiety. The hydrophobic residue is preferably a hydrocarbon chain having 8 to 28 carbon atoms, which may be saturated or unsaturated, linear or branched. With particular preference, this C8-C28 alkyl chain is linear.

Anionic surfactants will be understood to mean surfactants having exclusively anionic charges; they include for example carboxyl groups, sulfonic acid groups or sulfate groups. Particularly preferred anionic surfactants are alkyl sulfates, alkyl ether sulfates, acyl glutamates and C8-24 carboxylic acids and salts thereof, the so-called soaps. Cationic surfactants will be understood to mean surfactants having exclusively cationic charges; they include for example quaternary ammonium groups. Preference is given to cationic surfactants of the quaternary ammonium compound, esterquat and amidoamine type. Preferred quaternary ammonium compounds are ammonium halides and the imidazolium compounds known under the INCI names Quaternium-27 and Quaternium-83. Other cationic surfactants which can be used according to the invention are the quaternized protein hydrolyzates. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. The amphoteric surfactants are divided into ampholytic surfactants and zwitterionic surfactants. Ampholytic surfactants will be understood to mean those surface-active compounds which possess both acidic (for example —COOH or —SO₃H groups) and basic hydrophilic groups (for example amino groups) and therefore behave in an acidic or basic manner depending on conditions. Zwitterionic surfactants will be understood by a person skilled in the art to mean surfactants which carry both a negative and a positive charge in the same molecule. Examples of preferred zwitterionic surfactants are betaines, N-alkyl-N,N-dimethyl ammonium glycinates, N-acylaminopropyl-N,N-dimethylammonium glycinates and 2-alkyl-3-carboxymethyl-3-hydroxyethylimidazolines, having in each case 8 to 24 carbon atoms in the alkyl group. Examples of preferred ampholytic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyl taurines N-alkyl sarcosines, 2-alkylaminopropionic acids and alkylaminoacetic acids having in each case 8 to 24 carbon atoms in the alkyl group.

The compositions according to the invention, which are formulated as an emulsion, in particular as an oil-in-water emulsion, preferably include at least one nonionic oil-in-water emulsifier having an HLB value of more than 7 to 20. These are emulsifiers which are generally known to a person skilled in the art, as listed for example in Kirk-Othmer, “Encyclopedia of Chemical Technology”, 3rd edition, 1979, Volume 8, pages 913-916. For ethoxylated products, the HLB value is calculated according to the formula HLB=(100−L):5, where L is the proportion by weight of the lipophilic groups, that is to say of the fatty alkyl or fatty acyl groups, in the ethylene oxide adducts, expressed in percent by weight. In this connection, it may be preferred according to the invention if a water-in-oil emulsifier having an HLB value of greater than 1.0 and less than/equal to 7.0 is also used. Nonionic oil-in-water emulsifiers and nonionic water-in-oil emulsifiers which are suitable in the context of the present invention are described for example in German laid-open specification DE 102006004957.

In order to thicken the antiperspirant cosmetic agents according to the invention, use is preferably made of hydrogel-forming substances which are selected from cellulose ethers, especially hydroxyalkyl celluloses, in particular hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose, and also xanthan gum, sclerotium gum, succinoglucans, polygalactomannans, in particular guar gums and locust bean gum, in particular guar gum and locust bean gum itself and the nonionic hydroxyalkyl guar derivatives and locust bean gum derivatives, such as hydroxypropyl guar, carboxymethylhydroxypropyl guar, hydroxypropylmethyl guar, hydroxyethyl guar and carboxymethyl guar, also pectins, agar, carrageenan, tragacanth, gum arabic, karaya gum, tara gum, gellan, gelatin, casein, propylene glycol alginate, alginic acids and salts thereof, in particular sodium alginate, potassium alginate and calcium alginate, also polyvinylpyrrolidones, polyvinyl alcohols, polyacrylamides, also—although less preferred—physically modified (for example by pregelatinization) and/or chemically modified starches, in particular hydroxypropylated starch phosphates and octenyl starch succinates and the aluminum, calcium or sodium salts thereof, also—again less preferred—acrylic acid/acrylate copolymers, acrylic acid/acrylamide copolymers, acrylic acid/vinylpyrrolidone copolymers, acrylic acid/vinyl formamide copolymers, and polyacrylates. Particularly preferred hydrogel formers are selected from cellulose ethers, especially from hydroxyalkyl celluloses, in particular from hydroxypropyl cellulose, hydroxypropyl methyl cellulose, hydroxyethyl cellulose, carboxymethyl cellulose, cetyl hydroxyethyl cellulose, hydroxybutyl methyl cellulose and methyl hydroxyethyl cellulose, and mixtures thereof. Preferably hydroxyethyl cellulose is used as the hydrogel former.

To further aid the blocking of the efferent ducts of the sweat glands, it may be advantageous to add to the antiperspirant cosmetic agents according to the invention at least one chelating agent which is selected from ethylenediaminetetraacetic acid (EDTA) and salts thereof and nitrilotriacetic acid (NTA) and mixtures of said substances, in a total amount of from 0.01 to 0.5% by weight, preferably from 0.02 to 0.3% by weight, in particular from 0.05 to 0.1% by weight, based on the total weight of the antiperspirant agent according to the invention.

Although one advantage of the present invention lies in that, besides aluminum sesquichlorohydrate and triethyl citrate, no further antiperspirant active ingredients and/or deodorant active ingredients need be included therein, the antiperspirant effect of the antiperspirant cosmetic agents according to the invention can be increased in embodiments of the invention if at least one deodorant active ingredient is included in a total amount of from 0.0001 to 40% by weight, preferably from 0.2 to 20% by weight, more preferably from 1 to 15% by weight, in particular from 1.5 to 5% by weight, based on the total weight of the antiperspirant cosmetic agent according to the invention. If ethanol is used in the agents according to the invention, in the context of the present invention this is regarded not as a deodorant active ingredient but rather as a constituent of the carrier. Deodorant active ingredients which are preferred according to the invention are selected from the group consisting of (i) silver salts; (ii) aromatic alcohols, in particular 2-benzylheptan-1-ol and mixtures of 2-benzylheptan-1-ol and phenoxyethanol; (iii) 1,2-alkanediols having 5 to 12 carbon atoms, but preferably not ethylhexylglycerin; (iv) active ingredients against exoesterases, in particular against arylsulfatase, lipase, beta-glucuronidase and cystathionine-[beta]-lyase; (v) cationic phospholipids; (vi) odor absorbers, in particular silicates, such as montmorillonite, kaolinite, illite, beidellite, nontronite, saponite, hectorite, bentonite, smectite and talc, zeolites, zinc ricinoleate, cyclodextrins; (vii) deodorizing ion exchangers; (viii) germ inhibitors; (ix) prebiotic components; and (x) mixtures of said deodorant active ingredients.

Preferred antiperspirant cosmetic agents according to the invention further include at least one water-soluble polyvalent C₂₋₉ alkanol having 2 to 6 hydroxyl groups and/or at least one water-soluble polyethylene glycol having 3 to 50 ethylene oxide units, and mixtures thereof. These do not include the aforementioned deodorant active ingredients in the form of 1,2-alkanediols. Preferred alkanols are selected from 1,2-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomeric mixtures of cis- and trans-1,4-dimethylolcyclohexane, and mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, preference being given to PEG-3 to PEG-8.

According to another embodiment of the present invention, the antiperspirant cosmetic agents further include at least one skin-cooling active ingredient. Skin-cooling active ingredients which are suitable according to the invention are for example menthol, isopulegol and menthol derivatives, for example menthyl lactate, menthyl glycolate, menthyl ethyl oxamate, menthylpyrrolidonecarboxylic acid, menthyl methyl ether, menthoxypropanediol, menthone glycerol acetal (9-methyl-6-(1-methylethyl)-1,4-dioxaspiro (4.5)decane-2-methanol), monomenthyl succinate, 2-hydroxymethyl-3,5,5-trimethylcyclohexanol and 5-methyl-2-(1-methylethyl)cyclohexyl-N-ethyl oxamate. Preferred skin-cooling active ingredients are menthol, isopulegol, menthyl lactate, menthoxypropanediol, menthylpyrrolidonecarboxylic acid and 5-methyl-2-(1-methylethyl)cyclohexyl-N-ethyl oxamate and mixtures of said substances, in particular mixtures of menthol and menthyl lactate, menthol, menthol glycolate and menthyl lactate, menthol and menthoxypropanediol, or menthol and isopulegol.

It may also be provided that the antiperspirant cosmetic agents according to the invention include a propellant. In this case, they are prepared as an aerosol driven by a propellant gas. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane and tetrafluoropropene, both individually and in mixtures thereof. Hydrophilic propellant gases, such as carbon dioxide for example, can advantageously also be used in the context of the present invention if the proportion of hydrophilic gases is selected to be low and lipophilic propellant gas (for example propane/butane) is present in excess. Particular preference is given to propane, n-butane, isobutane and mixtures of said propellant gases. It has been found that the use of n-butane as the sole propellant gas may be particularly preferred according to the invention. The total amount of propellant is from 20 to 95% by weight, preferably from 30 to 85% by weight, in particular from 40 to 75% by weight, in each case based on the total weight of the antiperspirant consisting of the antiperspirant cosmetic agent according to the invention and the propellant.

According to the invention, lipophilic thickeners can also be used as auxiliaries. Preferably, the at least one antiperspirant aluminum salt is suspended undissolved in at least one cosmetic oil which is liquid at 20° C. and 1013 hPa. For better application, at least one lipophilic thickener may also be added to this suspension as a suspending aid. Lipophilic thickeners which are preferred according to the invention are selected from hydrophobized clay minerals and pyrogenic silicas.

In one preferred embodiment, the agents according to the invention include as a deodorizing active ingredient at least one aromatic alcohol of structure (AA-1),

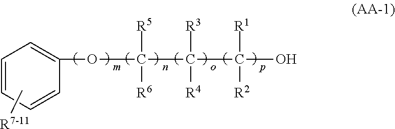

in which the residues R¹ to R⁶ independently of one another are a hydrogen atom, an alkyl group having 1 to 10 carbon atoms which may be linear or branched and may be substituted with OH groups or alkoxy groups having 1 to 5 carbon atoms, or an alkenyl group having 2 to 10 carbon atoms which may be linear or branched and may be substituted with OH groups or alkoxy groups having 1 to 5 carbon atoms,
the residues R⁷ to R¹¹ independently of one another are a hydrogen atom, a halogen atom, in particular a chlorine atom, or an alkyl group having 1 to 10 carbon atoms which may be linear or branched and may be substituted with OH groups or alkoxy groups having 1 to 5 carbon atoms, in particular with a methoxy group,
m=0 or 1, n, o, p independently of one another are integers from 0 to 10, where at least one of the values n, o, p≠0.

Particularly preferred products according to the invention include at least one alcohol AA-1, as described above, which is selected from phenoxyethanol, anise alcohol, 2-methyl-5-phenylpentan-1-ol, 1,1-dimethyl-3-phenylpropan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2,2-dimethyl-3-phenylpropan-1-ol, 2,2-dimethyl-3-(3′-methylphenyl)propan-1-ol, 2-ethyl-3-phenylpropan-1-ol, 2-ethyl-3-(3′-methylphenyl)propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-ol, 2-ethyl-3-(2′-methylphenyl)propan-1-ol, 2-ethyl-3-(4′-methylphenyl)propan-1-ol, 3-(3′,4′-dimethylphenyl)-2-ethylpropan-1-ol, 2-ethyl-3-(4′-methoxyphenyl)propan-1-ol, 3-(3′,4′-dimethoxyphenyl)-2-ethylpropan-1-ol, 2-allyl-3-phenylpropan-1-ol and 2-n-pentyl-3-phenylpropan-1-ol, and mixtures thereof. Particular preference is given to phenoxyethanol and mixtures of 2-benzylheptan-1-ol and phenoxyethanol. Other particularly preferred compositions according to the invention include at least one alcohol AA-1, as described above, in a total amount of from 0.05 to 10% by weight, preferably from 0.1 to 5% by weight, particularly preferably from 0.2 to 2% by weight, extremely preferably from 0.3 to 1.5% by weight, in each case based on the weight of the composition without taking into account any propellant that may be present.

Particularly preferred antiperspirant agents according to the invention further include at least one fragrance. The definition of a fragrance in the context of the present invention coincides with the definition that is customary for a person skilled in the art, as can be found in RÖMPP Chemie Lexikon, December 2007. According to said definition, a fragrance is a chemical compound having a smell and/or taste which stimulates the receptors of the hair cells of the olfactory system (adequate stimulus). The physical and chemical properties necessary for this are a low molar mass of at most 300 g/mol, a high vapor pressure, minimal water solubility and high lipid solubility, weak polarity, and the presence of at least one osmophoric group in the molecule. In order to delineate volatile, low-molecular-weight substances, which usually and also in the context of the present invention are regarded and used not as a fragrance but primarily as a solvent, such as for example ethanol, propanol, isopropanol and acetone, from fragrances according to the invention, fragrances according to the invention have a molar mass of from 74 to 300 g/mol, include at least one osmophoric group in the molecule and have a smell and/or taste, that is to say they stimulate the receptors of the hair cells of the olfactory system.

As fragrances, use can be made of perfumes, perfume oils or perfume oil constituents. According to the invention, perfume oils or scents may be individual fragrance compounds, for example synthetic products of the ester, ether, aldehyde, ketone, alcohol and hydrocarbon type. Fragrance compounds of the ester type are for example benzyl acetate, phenoxyethyl isobutyrate, p-tert-butyl cyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, floramat, melusat and jasmecyclat. The ethers include for example benzyl ethyl ether and Ambroxan; the aldehydes include for example the linear alkanals having 8-18 C atoms, citral, citronellal, citronellyl oxyacetaldehyde, cyclamen aldehyde, lilial and bourgeonal; the ketones include for example the ionones, alpha-isomethyl ionone and methyl cedryl ketone; the alcohols include anethol, citronellol, eugenol, geraniol, linalool, phenylethyl alcohol and terpineol; and the hydrocarbons include primarily the terpenes such as limonene and pinene. Preferably, however, use will be made of mixtures of different fragrances which together produce a pleasant scent note.

Particularly preferred antiperspirant agents according to the invention include at least one fragrance in a total amount of from 0.00001 to 10% by weight, preferably from 0.5 to 7% by weight, extremely preferably from 1 to 6% by weight, in each case based on the total weight of the agent without taking into account any propellant that may be present.

The agents according to the invention include the ingredients and/or active substances in a cosmetically acceptable carrier.

Preferred cosmetically acceptable carriers are aqueous, alcoholic or aqueous-alcoholic media including preferably at least 10% by weight water, calculated on the total weight of the agent.

With particular preference, the cosmetic carrier according to the invention includes water, in particular in the amount that the cosmetic agent, calculated on the total weight of the agent, includes at least 10% by weight, in particular at least 20.0% by weight, most preferably at least 30% by weight water.

The alcohols used may be in particular the lower alcohols having 1 to 4 carbon atoms which are usually used for cosmetic purposes, such as for example ethanol and isopropanol.

Examples of water-soluble solvents as a cosolvent are glycerol and/or ethylene glycol and/or 1,2-propylene glycol in an amount of from 0 to 30% by weight, based on the total agent.

The antiperspirant cosmetic agent according to the invention may be applied by various methods. According to one preferred embodiment, the antiperspirant cosmetic agent is prepared as a spray application. The spray application takes place by means of a spray device which includes in a container a filling consisting of the liquid, viscous-flowable, suspension-like or powdered antiperspirant cosmetic agent according to the invention. The filling may be under the pressure of a propellant (pressurized gas cans, pressurized gas packaging, aerosol packaging), or it may be a mechanically operated pump atomizer without propellant gas (pump sprays/squeeze bottles). The containers have a withdrawal device, preferably in the form of valves, which enable the contents to be withdrawn in the form of a mist, puff, foam, powder, paste or liquid jet. Suitable containers for spray devices are especially cylindrical vessels made of metal (aluminum, tinplate, volume preferably at most 1000 mL), protected or shatterproof glass or plastic (volume preferably at most 220 mL) or shattering glass or plastic (volume preferably 50 to 400 mL). Agents in cream, gel, paste and liquid form may be packaged for example in pump dispensers, spray dispensers or squeeze dispensers, in particular including in multi-chamber pump dispensers, multi-chamber spray dispensers or multi-chamber squeeze dispensers. The packaging for the agents according to the invention may be opaque but also transparent or translucent.

The antiperspirant cosmetic agent is preferably prepared as a stick, soft solid, cream, roll-on, dibenzylidene alditol-based gel, or loose or compact powder. The formulation of the antiperspirant cosmetic agents according to the invention in a particular administration form, such as for example an antiperspirant roll-on, an antiperspirant stick or an antiperspirant gel, preferably depends on the requirements of the intended use. Therefore, depending on the intended use, the antiperspirant cosmetic agents according to the invention may be in solid, semi-solid, liquid, disperse, emulsified, suspended, gel-like, multiphase or powdered form. In the context of the present invention, the term liquid also encompasses any types of solid dispersions in liquids. Furthermore, in the context of the present invention, multiphase antiperspirant cosmetic agents according to the invention will be understood to mean agents which comprise at least 2 different phases with phase separation, and in which the phases may be arranged horizontally, that is to say one above the other, or vertically, that is to say one next to the other.

Application may take place for example by means of a roller ball applicator. Such rollers comprise a ball mounted in a ball bed, which ball can be moved by movement over a surface. In doing so, the ball takes up some of the antiperspirant cosmetic agent according to the invention that is to be distributed, and conveys it to the surface to be treated. As mentioned above, the packaging for the agents according to the invention may be opaque, transparent or translucent.

Furthermore, it is also possible to apply the antiperspirant cosmetic agents according to the invention by means of a solid stick in the form of a solid emulsion.

However, it may also be preferred according to the invention that the antiperspirant cosmetic agent is included on and/or in a disposable substrate, selected from the group consisting of wipes, pads and wads. Particular preference is given to moist wipes, that is to say moist wipes which have been pre-prepared for the user and which are preferably packaged individually, as are well known for example from the field of glass cleaning or from the field of moist toilet tissues. Such moist wipes, which advantageously may also include preservatives, have an antiperspirant cosmetic agent according to the invention impregnated therein or applied thereto and are preferably packaged individually. They may be used for example as a deodorant wipe, which is particularly beneficial for use on the go. Preferred substrate materials are selected from porous flat wipes. They may be made of a fibrous or cellular flexible material which has sufficient mechanical stability and at the same time softness for use on the skin. These wipes include wipes made of woven and nonwoven synthetic and natural fibers, felt, paper or foam, such as hydrophilic polyurethane foam. Deodorizing or antiperspirant substrates which are preferred according to the invention can be obtained by soaking or impregnation or also by melting an antiperspirant cosmetic agent according to the invention onto a substrate.

Overview in Table Form

The composition of some preferred cosmetic agents can be seen in the following tables (unless indicated otherwise, figures specified in % by weight relate in each case to the total weight of the cosmetic agent without taking into account any propellant that may be used).

| | Formula 1 | Formula 2 | Formula 3 | Formula 4 |
|---|---|---|---|---|
| Triethyl citrate | 0.1 to 7.0 | 0.5 to 5.0 | 0.5 to 3.0 | 1.0 to 2.0 |
| Aluminum | 5.0 to 15.0 | 6.0 to 14.0 | 7.0 to 13.0 | 8.0 to 12.0 |
| sesquichlorohydrate | | | | |
| Misc | ad 100 | ad 100 | ad 100 | ad 100 |
| | Formula 1a | Formula 2a | Formula 3a | Formula 4a |
| Triethyl citrate | 0.1 to 7.0 | 0.5 to 5.0 | 0.5 to 3.0 | 1.0 to 2.0 |
| Aluminum | 5.0 to 15.0 | 6.0 to 14.0 | 7.0 to 13.0 | 8.0 to 12.0 |
| sesquichlorohydrate | | | | |
| Chelating agent, e.g. EDTA | 0.01 to 0.5 | 0.02 to 0.4 | 0.03 to 0.3 | 0.05 to 0.1 |
| Misc | ad 100 | ad 100 | ad 100 | ad 100 |
| | Formula 1b | Formula 2b | Formula 3b | Formula 4b |
| Triethyl citrate | 0.1 to 7.0 | 0.5 to 5.0 | 0.5 to 3.0 | 1.0 to 2.0 |
| Aluminum | 5.0 to 15.0 | 6.0 to 14.0 | 7.0 to 13.0 | 8.0 to 12.0 |
| sesquichlorohydrate | | | | |
| Phenoxyethanol | 0.05 to 10.0 | 0.1 to 3.0 | 0.3 to 2.0 | 0.5 to 1.5 |
| Misc | ad 100 | ad 100 | ad 100 | ad m100 |
| | Formula 1c | Formula 2c | Formula 3c | Formula 4c |
| Triethyl citrate | 0.1 to 7.0 | 0.5 to 5.0 | 0.5 to 3.0 | 1.0 to 2.0 |
| Aluminum | 5.0 to 15.0 | 6.0 to 14.0 | 7.0 to 13.0 | 8.0 to 12.0 |
| sesquichlorohydrate | | | | |
| Chelating agent, e.g. EDTA | 0.01 to 0.5 | 0.02 to 0.4 | 0.03 to 0.3 | 0.05 to 0.1 |
| Phenoxyethanol | 0.05 to 10.0 | 0.1 to 3.0 | 0.3 to 2.0 | 0.5 to 1.5 |
| Misc | ad 100 | ad 100 | ad 100 | ad 100 |

According to the invention, “Misc” is to be understood to be a cosmetic carrier, and possibly other conventional constituents of antiperspirant agents. Embodiments in which “Misc” includes no ethylhexylglycerin, and embodiments in which “Misc” includes no dipropylene glycol, and embodiments in which “Misc” includes no further antiperspirant active ingredients, and embodiments in which “Misc” does not include one or more of ethylhexylglycerin, dipropylene glycol and further antiperspirant active ingredients, are also expressly encompassed here.

### Exemplary Embodiments

The following formulation examples are intended to illustrate the subject matter of the invention without limiting the latter thereto.

The cosmetic compositions described in the table below were prepared as emulsions.

Antiperspirant roll-ons (figures specified in % by weight)

| Component or INCI | Manufacturer | 1 | 2 | 3 | 4 |
|---|---|---|---|---|---|
| PPG-15 Stearyl | | 0.5 | 0.5 | 0.5 | 0.5 |
| Ether | | | | | |
| Steareth-2 | | 2.4 | 2.4 | 2.4 | 2.4 |
| Steareth-21 | | 1.5 | 1.5 | 1.5 | 1.5 |
| Triethyl Citrate | Vertellus | 1.0 | 1.0 | 1.5 | 2.0 |
| (citroflex 2) | Specialities | | | | |
| | Inc. | | | | |
| Aluminum, | | 20.0 | 25.0 | 30.0 | 30.0 |
| Sesquichlorohydrate | | | | | |
| (Reach 301L, 40% | | | | | |
| in water) | | | | | |
| EDTA BX Powder | BASF | 0.1 | 0.1 | 0.1 | 0.1 |
| Perfume | | 1.0 | 1.0 | 1.0 | 1.0 |
| Water | | 73.5 | 68.5 | 64.0 | 63.5 |

Antiperspirant aerosols (figures specified in % by weight)

| Component or INCI | Manufacturer | 5 | 6 | 7 |
|---|---|---|---|---|
| Cyclopentasiloxane (Xiameter | Dow Corning | 10.7 | 10.7 | 10.7 |
| PMX-0245) | | | | |
| 25% PEG/PPG-18/18 | Dow Corning | 5.6 | 5.6 | 5.6 |
| Dimethicone/85% | | | | |
| Dimethicone 5 cts (Dow | | | | |
| Corning ES-5277 DM) | | | | |
| Isopropyl Myristate | BASF | 6.6 | 6.6 | 6.6 |
| Propylene glycol | | 23.4 | 23.4 | 23.4 |
| Phenoxyethanol | | 0.5 | 0.5 | 0.5 |
| Aluminum, Sesquichlorohydrate | Summit | 20.0 | 25.0 | 30.0 |
| (Reach 301L, 40% in water) | Reheis | | | |
| Triethyl Citrate (citroflex 2) | Vertellus | 1.0 | 1.0 | 2.0 |
| | Specialities | | | |
| | Inc. | | | |
| Perfume | | 2.5 | 2.5 | 2.5 |
| Water | | 29.7 | 24.7 | 18.7 |

Formulations 5, 6 and 7 were filled into aerosol cans in a weight ratio of 1:4 with the propellant propane/butane (15/85).

The pH of cosmetic compositions 1 to 7 according to the invention, measured under normal conditions using the pH meter pH730 pH from InoLab, was in each case between 3.5 and 4.0.

Results:

When using the formulations, test subjects observed very good skin compatibility, few residues and stains on the skin and fabrics, and an improved, less sticky feel on the skin.

Furthermore, when the cosmetic agent according to the invention (Formulation E below) was applied to the skin surface, a lowering of the pH of the skin was observed. The comparative formulation (V) comprising 26% aluminum chlorohydrate without triethyl citrate does not exhibit any measurable lowering of the pH in this test design. The formulations were applied to the underarms of 20 test subjects under controlled conditions. In the 5 days beforehand, no deodorant products were applied and the underarms were washed only with neutral soap. The measurements took place using the SKIN pH meter 905 (Courage+Khazaka electronic GmbH). The two products were tested on different subject collectives. It was therefore possible to compare only the pH shift, but not the absolute values at the respective time.

Effect of different test formulations on the pH of the skin of the underarms

| Component or INCI | Manufacturer | E | V |
|---|---|---|---|
| PPG-15 Stearyl Ether | | 0.5 | 0.5 |
| Steareth-2 | | 2.4 | 2.4 |
| Steareth-21 | | 1.5 | 1.5 |
| Triethyl Citrate | Vertellus | 1.0 | — |
| (citroflex 2) | Specialities | | |
| | Inc. | | |
| Aluminum, Sesquichlorohydrate | Summit | 25.0 | — |
| (Reach 301L, 40% in water) | Reheis | | |
| Aluminum Chlorohydrate | Clariant | — | 26.0 |
| (Locron L, 50% in water) | | | |
| EDTA BX Powder | BASF | 0.1 | 0.1 |
| Water | | 69.5 | 69.5 |
| Auxiliary Skin pH | T 0 min | 6.0 | 5.3 |
| | T 20 min | 5.8 | 5.3 |
| | T 40 min | 5.8 | 5.4 |
| | T 60 min | 5.7 | 5.4 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent in the form of an aqueous emulsion, comprising:
aluminum sesquichlorohydrate and
triethyl citrate,
wherein the cosmetic agent has a pH of from 3.5 to 4.0.

2. The cosmetic agent according to claim 1, wherein the aluminum sesquichlorohydrate is included in an amount of from 5.0 to 15.0% by weight and the triethyl citrate is included in an amount of from 0.1 to 7% by weight, wherein the figures specified in % by weight relate in each case to the total weight of the cosmetic agent, without taking into account any propellant that may be present.

3. The cosmetic agent according to claim 1, wherein the aluminum sesquichlorohydrate is included in an amount of from 8.0 to 12.0% by weight and the triethyl citrate is included in an amount of from 0.5 to 2.0% by weight, wherein the figures specified in % by weight relate in each case to the total weight of the cosmetic agent, without taking into account any propellant that may be present.

4. The cosmetic agent according to claim 1, wherein the cosmetic agent includes no ethylhexylglycerin.

5. The cosmetic agent according to claim 1, wherein the cosmetic agent includes no dipropylene glycol.

6. The cosmetic agent according to claim 1, wherein the aluminum sesquichlorohydrate has a molar Al/Cl ratio of from 1.5:1 to 1.8:1.

7. The cosmetic agent according to claim 1, wherein the cosmetic agent further includes phenoxyethanol.

8. The cosmetic agent according to claim 1, wherein the cosmetic agent is present as an oil-in-water emulsion.

9. A method for the non-therapeutic, cosmetic antiperspirant treatment of the body, comprising: applying an antiperspirant cosmetic agent according to claim 1 to a skin.
